(19) United States
(12) Patent Application Publication
(10) Pub. No.: US 2009/0264840 A1
(43) Pub. Date: Oct. 22, 2009

(54) Suction Device for Organic liquids form Absorbent Towels and Tampons and the Like for Hygienic and Sanitary Uses and for Liquids of Other Kind for Different Applications

(76) Inventor: Marconato Virginio, S. Pietro di Feletto (IT)

Correspondence Address: BUCKNAM AND ARCHER, 1077 NORTHERN BOULEVARD, ROSLYN, NY, 11576, US

(21) Appl. No.: 11/991,975

(22) PCT Filed: Nov. 14, 2007
(86) PCT No.: PCT/IB2007/003520

§ 371 Date: Mar. 13, 2008

(30) Foreign Application Priority Data
Nov. 27, 2006 (IT) PN2006A000097

(51) Int. Cl. A61M 1/00; A61F 13/20; A61F 13/47; A47L 5/00

(52) U.S. Cl. 604/319; 604/385.17; 604/385.01; 15/300.1

(57) ABSTRACT

Suction device for organic liquids from absorbent towels and tampons and the like for hygienic and sanitary uses and for liquids of other kind for different applications. Device comprising a container (6) which may be connected to each absorbent towel/tampon and provided with adjustment means (12), which may be connected to vacuum production means (16, 18, 21, 25, 26) and adapted to be displaced from an open position to a closing position, and vice versa, respectively to create a high vacuum condition and to maintain such vacuum condition in the interior of the container (6), said container (6) being adapted to be put into communication with the environment and the towel/tampon, by displacing said adjustment means (12) on the open position again, thereby producing a suction action of variable duration adapted to suck and to convey the liquids, absorbed by said absorbent towel/tampon, in the interior of the container (6), where they are collected and afterwards discharged outwards, and with subsequent arrangement of the container (6) to be put under vacuum condition again, with consequent further suctions and discharges of the liquids with respect to the same container.

The invention relates to a suction device for organic liquids from absorbent towels and tampons and the like for hygienic and sanitary uses and for liquids of other kind for different applications.

Absorbing systems for organic liquids for hygienic and sanitary uses are known, which are constituted by absorbent materials like absorbent towels, tampons and the like, which are applied on to the human body parts from which the organic liquids of different kind (urine, blood etc. . . . ) are emitted, and are removed when they are fully soaked with the same liquids, and substituted with further absorbent materials.

Generally, these absorbent materials are made of reduces sizes and tends to absorb and to be filled completely and quickly with the organic liquids, so that they must be removed and substituted frequently. However, sometimes it may happen that such absorbent towels and tampons are left for long times on to the persons, specially in the case of patients forced to remain still on bed after surgical operations, thereby remaining for a long time into contact with the skin of the same persons, and producing irritation and maceration of the skin.

The object of the present invention is to eliminate the drawbacks and limits and the above mentioned suction devices and systems employed at the present time, by a suction device for organic liquids from absorbent materials such as absorbent towels and tampons, which are applied on to the human body parts emitting such liquids, adapted to absorb the same liquids in a continuous manner, thereby reducing the need of a frequent change of the same absorbent materials, and keeping the body parts under dried and hygienic conditions, by avoiding that the above described harmful consequences on the skin occurs, as well as by eliminating the use of any suction apparatus.

Moreover, this suction device may be used also for suction of liquids of other kind for different applications. The device according to the invention is made with the constructive characteristics substantially described, with particular reference to the attached claims of the present patent.

The invention will be understood better from the following description, given by way of not limitative example and with reference to the accompanying drawings, wherein:

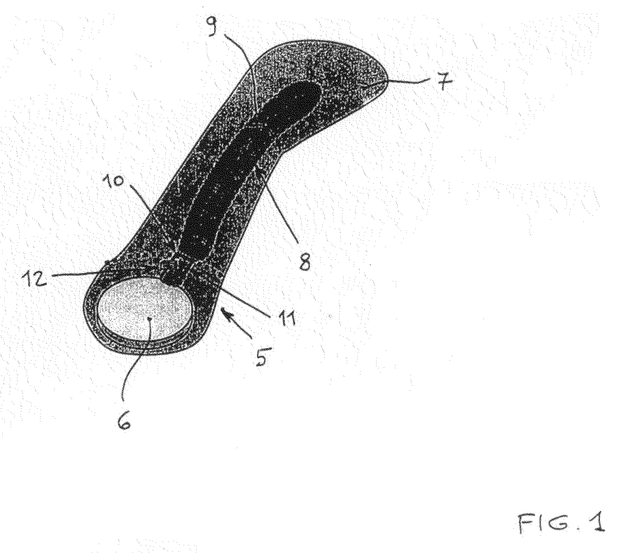

FIG. 1 shows a perspective front view of a suction device according to the invention, on a first constructive embodiment thereof;

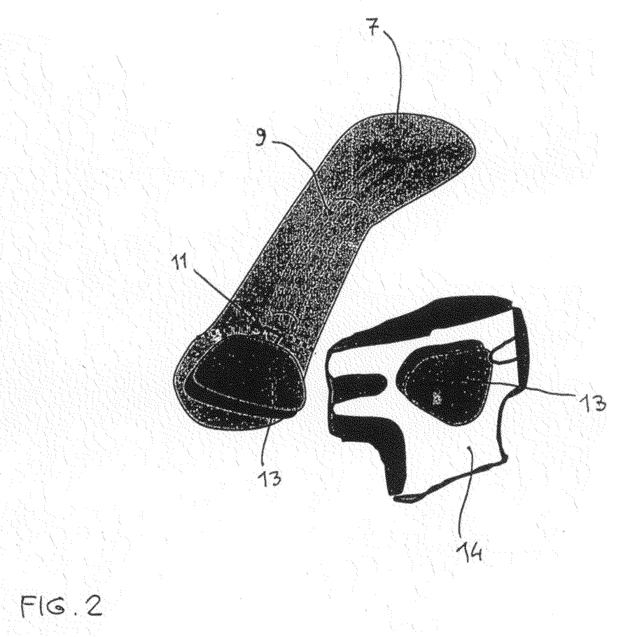

FIG. 2 shows a perspective front view of the suction device according to the invention, on a second constructive embodiment thereof;

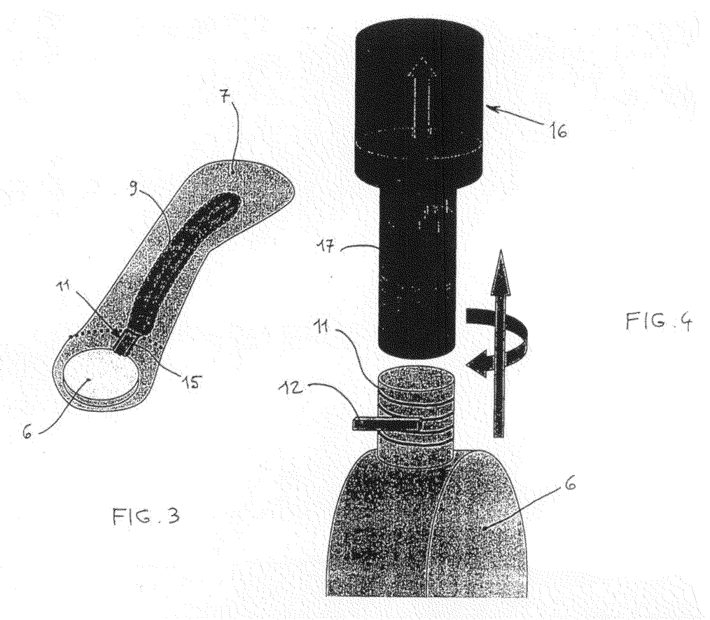

FIGS. 3 and 4 show a perspective front view of the suction device according to the invention, on two different preparation steps thereof for its application on to the human body;

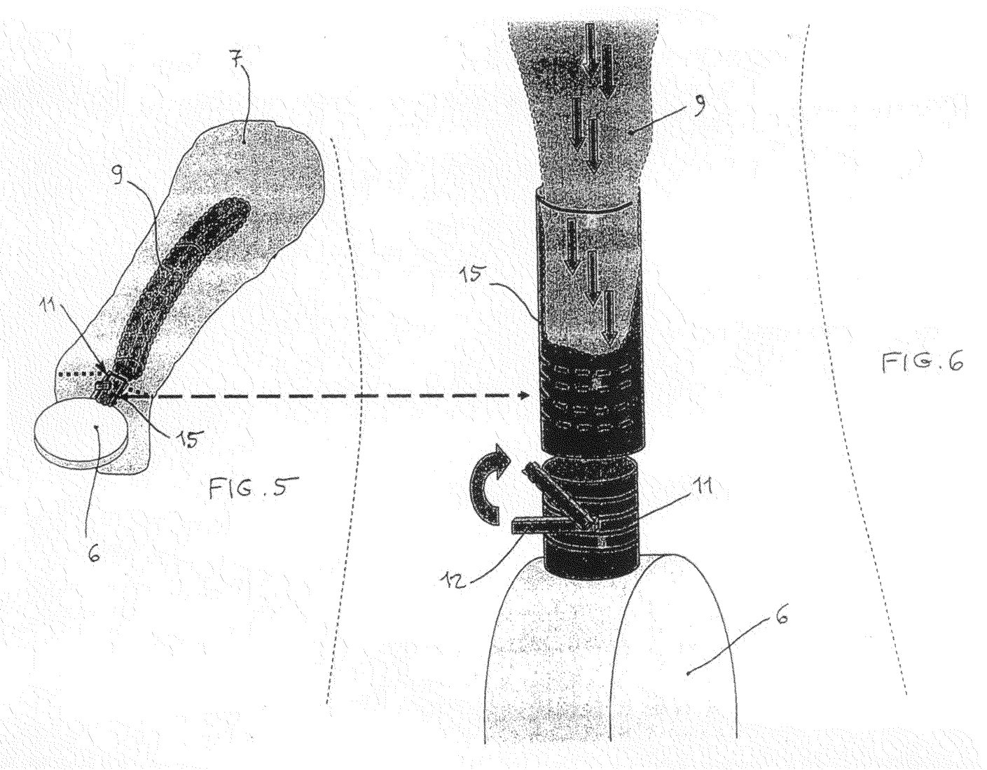

FIGS. 5 and 6 show a perspective front view of the suction device of FIGS. 3 and 4, on two additional preparation steps thereof;

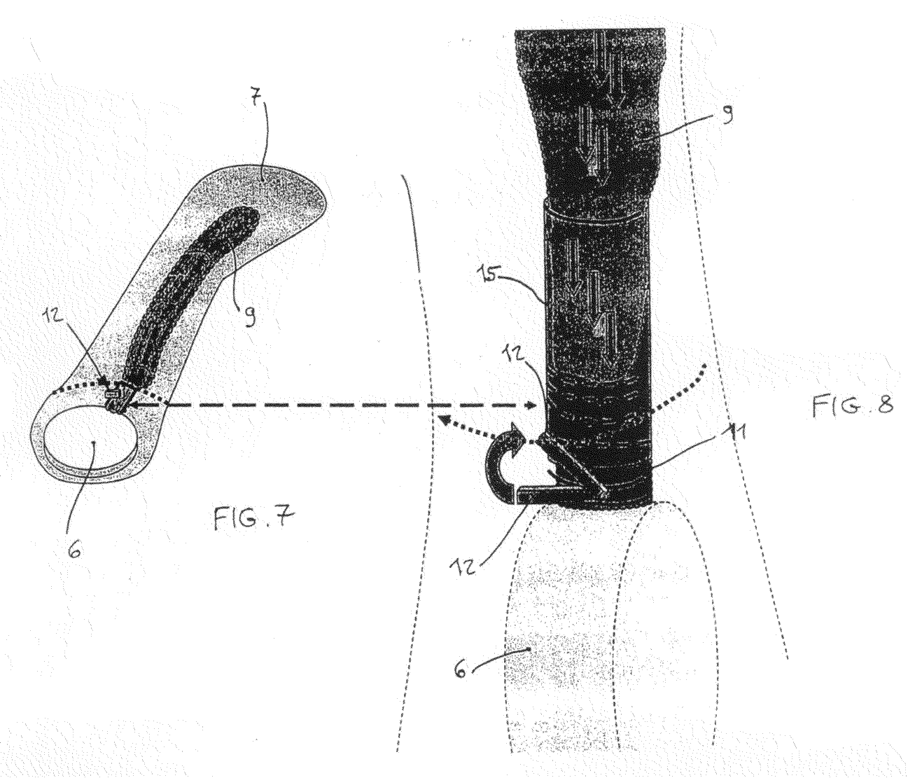

FIG. 7 shows a perspective front view of the suction device of FIGS. 3-6, on the application step on the human body thereof;

FIG. 8 shows a perspective front view of the suction device of FIG. 7, on the liquid suction step;

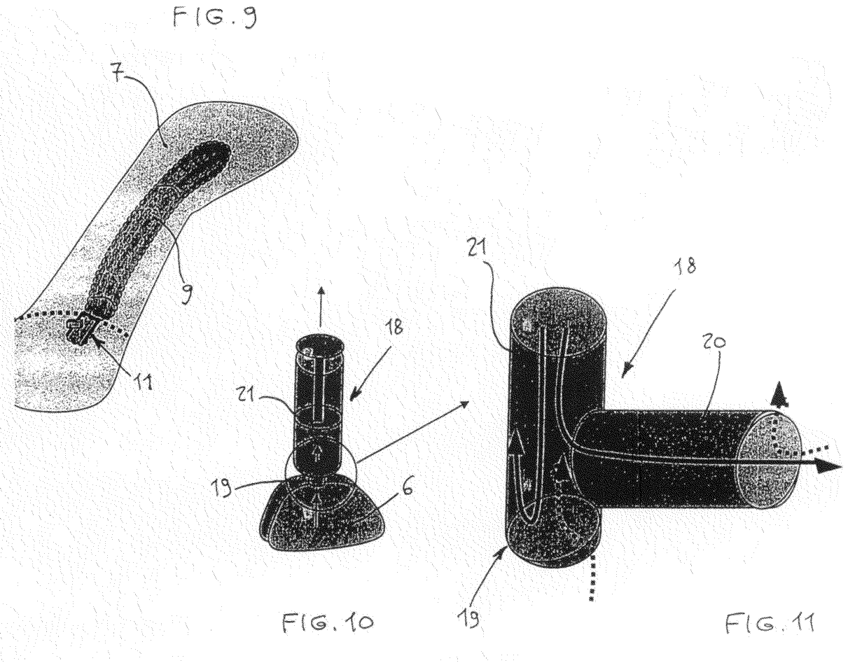

FIGS. 9, 10 and 11 show a perspective front view of a suction device of the type illustrated by FIG. 1, on different preparation steps for its application on the human body, according to a first preparation manner;

FIGS. 12, 13, 14 and 15 show a perspective front view of a suction device of the type illustrated by FIG. 1, on different preparation steps for its application on to the human body, according to another preparation manner;

FIG. 16 shows a perspective front view of the present suction device, on to another constructive embodiment thereof, and on the application step on to the human body;

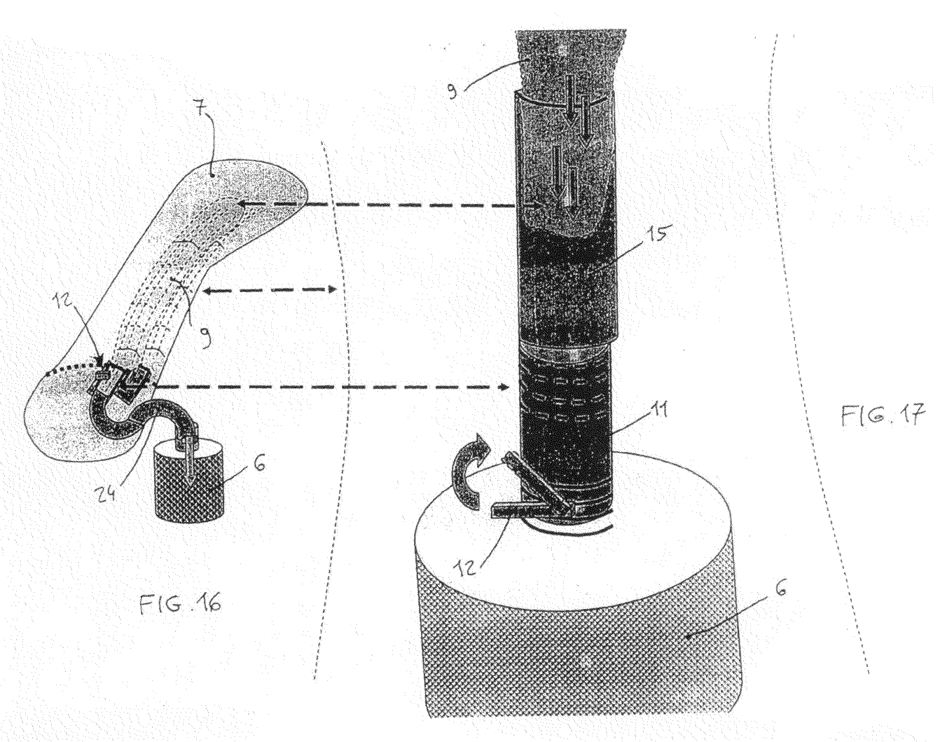

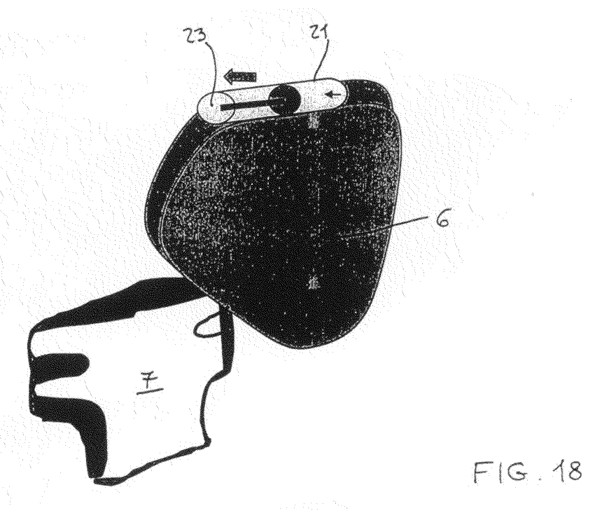

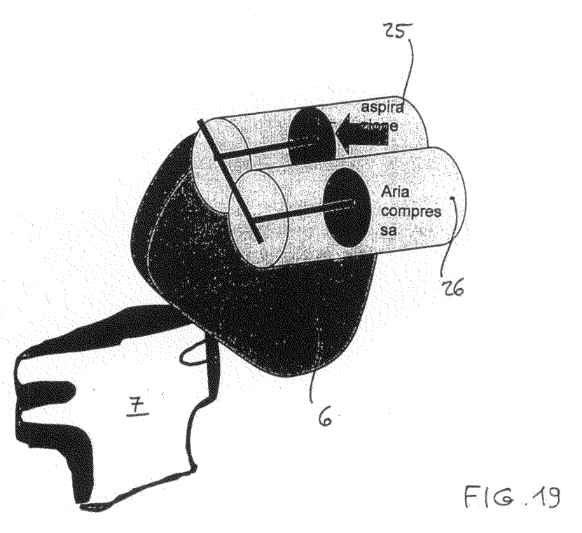

FIG. 17 shows a perspective front view of the suction device of FIG. 9, on the suction step of the emitted liquids;

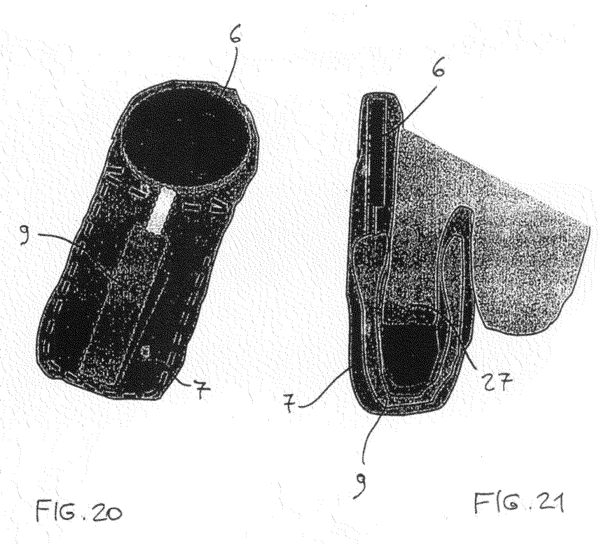

FIGS. 18 and 19 show a perspective front view of the suction device according to the invention, on two further constructive embodiments;

FIGS. 20 and 21 show a respective front and side view of a suction device according to the invention, applied on to a man, on a first application manner thereof;

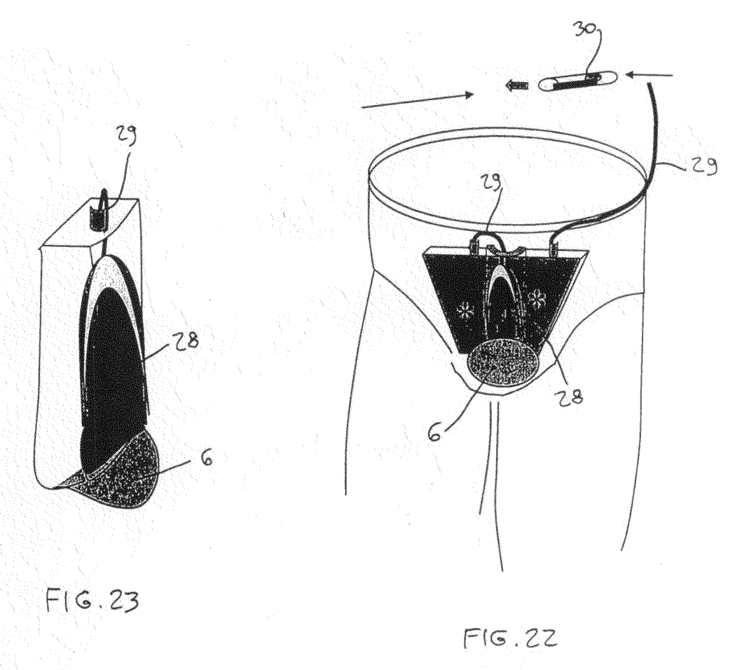

FIGS. 22 and 23 show a respective front and side view of a suction device according to the invention, applied on to a man, on a second application manner thereof;

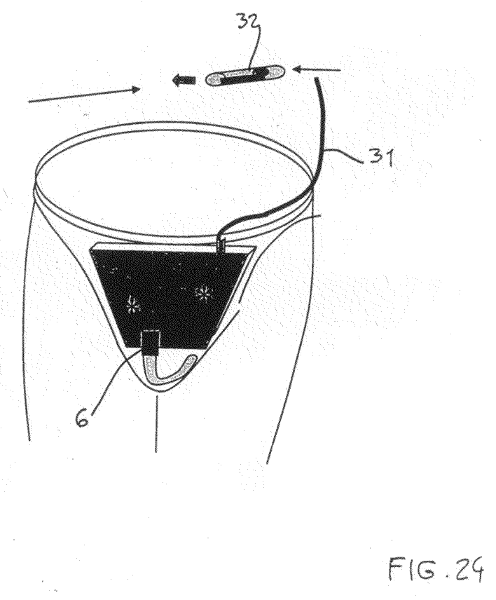

FIG. 24 shows a front view of a suction device according to the invention, applied on to a woman;

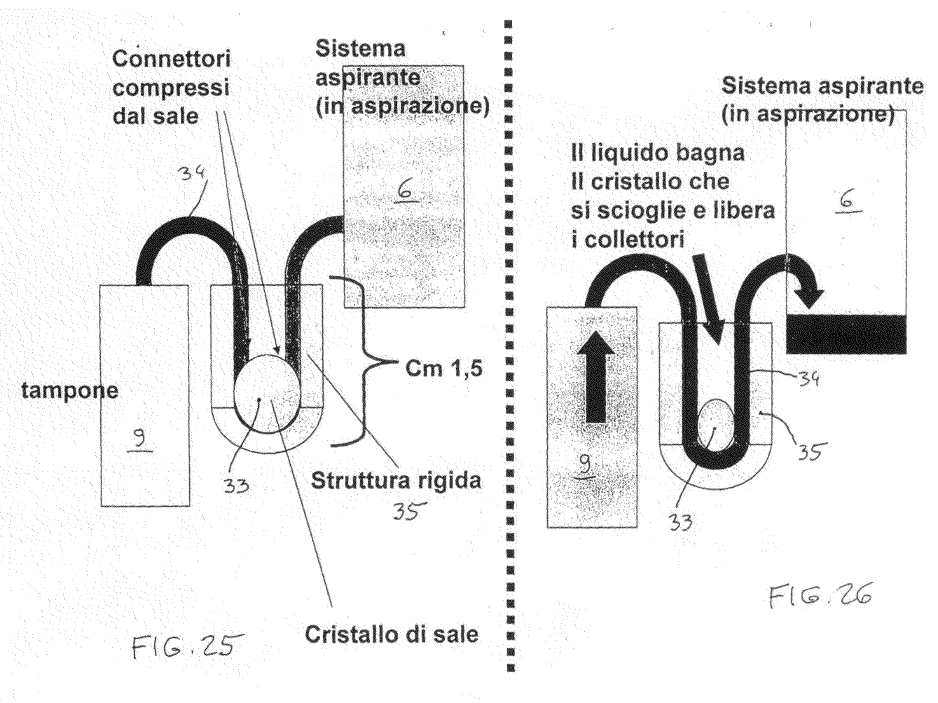

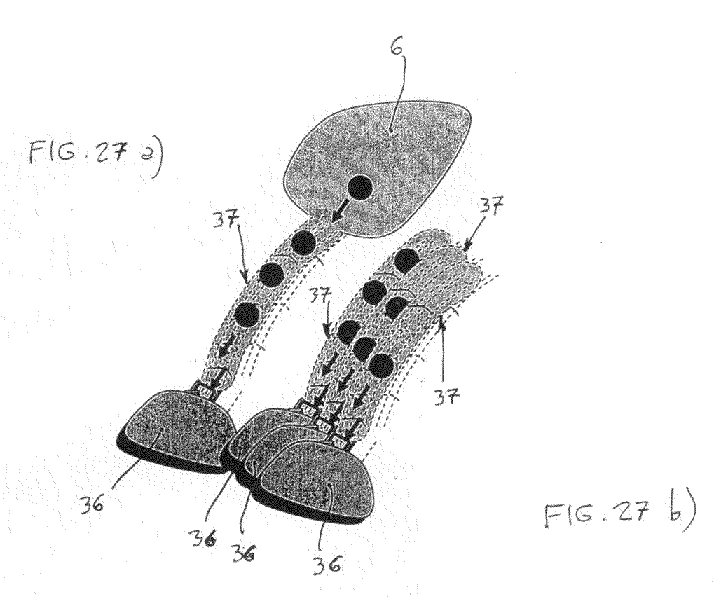

FIGS. 25 and 26 show a schematic view of a suction device according to the invention, on to an additional constructive embodiment and on two different operative steps;

FIG. 27 shows a perspective front view of a suction device according to the invention, used for a different application thereof;

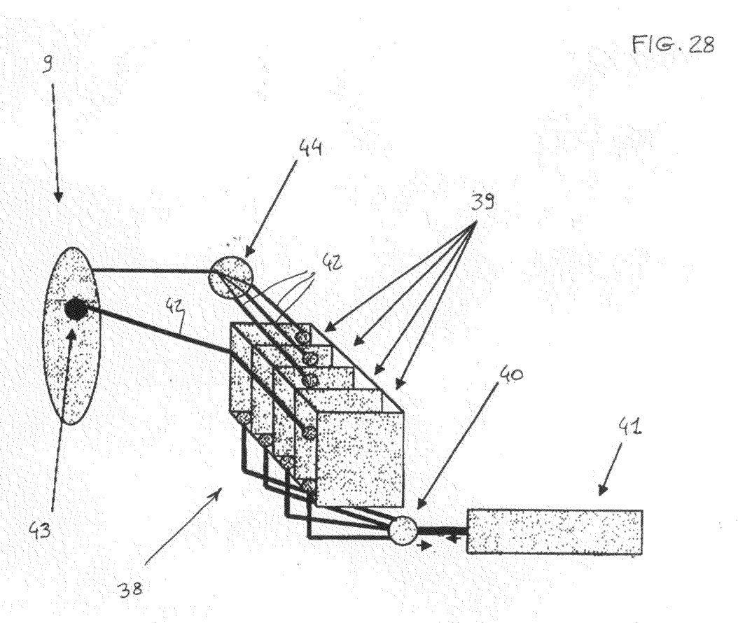

FIG. 28 shows schematically a particular constructive embodiment of the suction device according to the invention;

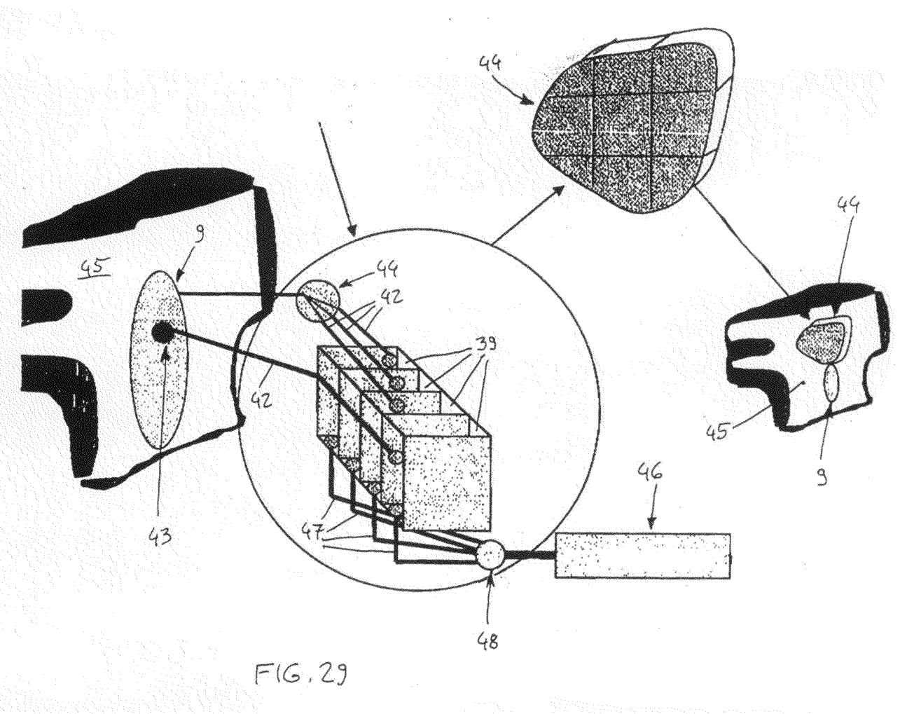

FIG. 29 shows schematically a further possible constructive embodiment of the suction device according to the invention.

The above Figures represent schematically a suction device for liquids of various kind (particularly but not exclusively organic liquids such as urine, blood, etc.), applied to absorbent materials of various kind contained into absorbent towels, tampons and the like. The device may be used in the hygienic-sanitary field (for instance, when it is applied on to living beings from whom the organic liquids are emitted), but it may also be used on other “hygienic-sanitary” application fields such as the building field, in the baths, room cleaning etc. The present absorbing and suction device is described hereinafter on various embodiments thereof, by way of example, which are focused to the application to the male and female human body, but of course it may be also used for applications of other kind, of which some possible applications thereof will be indicated by way of example.

With reference to the FIG. 1, in which a liquid suction device 5 according to the invention is shown, on a first embodiment thereof, it is noted that it is substantially constituted by at least a container 6 of more or less reduced sizes, into which a high vacuum is produced in advance (or periodically renewed), in order to provide for a suction action adapted to call back (and contain) the organic liquids absorbed by a tampon 9. Such container is introduced removably into at least an absorbent towel 7 with different shapes and sizes, depending on the applications to which it is assigned. Therefore, the container 6 is connected to the tampon 9 (whose housing 8 is in the towel portion being more affected by the liquid to be sucked) made of absorbent material and having a desired anatomical shape and size depending on the user's needs. Such tampon 9 is very compressed into the joining sleeve 11 with the container 6 under vacuum condition, and this in order to make easier and guarantee the suction of the liquid absorbed by the tampon. In this way, the liquid absorbed by the tampon 9 is “called back” in the interior of the container 6, which tampon is so “renewed” and may absorb additional liquid until the “suction force” of the container under vacuum condition is exhausted. Furthermore, also the vacuum condition in the interior of the container 6 may be renewed with various systems, by increasing the system potentiality. A cock 12 is interposed between the sleeve 11 of the tampon 9 and the container 6, which cock is adjustable from a closed position to a modulated open position thereof, and vice versa. In this way, the container 6 under vacuum condition (sucking) is put into communication with the absorbent system formed by the towel 7 and the tampon 9. In this way, in the case in which the cock 12 is closed, the container 6 does not come into contact with the environment and therefore the depression existing therein is maintained. Under the high vacuum condition, which is generated as it will be described in the interior of the container 6, with the closed cock, no effect is determined on the liquids which are absorbed by capillarity by the towel 7 and the tampon 9, so that these latter tend to be filled progressively with these liquids, by losing their capacity to keep dried and cleaned the affected body parts. Vice versa, in the second case of the cock 12 moved into the various open positions, the container 6 comes into contact with the tampon 9, so that the depression existing in the container determines a suction of the liquids which are called back by the towel 7 and the tampon 9, and from the tampon 9 in the interior of the container 6, with consequent progressive collection of the same liquids into the container 6. In this way, by adjusting adequately the level of the vacuum created into the container 6 and the time in which the existing depression of such container is annulled and kept to the level of the atmospheric pressure, it is possible to change the liquid suction intensity and duration, and therefore the quantity of liquid being absorbed by the towel 7 and the tampon 9 and introduced in the container 6. Consequently, while the container 6 continues to absorb the liquid from the towel 7 and the tampon 9, thereby discharging the liquid from these latter, such towel and tampon still continue to absorb additional liquid, for the entire duration in which a depression in the inner room of the container 6 does exist.

When the suction of the liquid by the container 6 is ended, this latter is removed from its position and the collected liquid is emptied therefrom, which liquid is discharged outside, and after that the high vacuum condition has been again created in the container 6, this latter is applied again in the previous position, with consequent further suction of the organic liquid from the towel 7 and the tampon 9. Thus, it appears evident that, thanks to the use of the suction device according to the invention, it is possible to keep the absorbent towel and tampon, which are into contact to the skin, under satisfactory dried and hygienic conditions for times longer than those of the towels and tampons used at the present time, with consequent advantages in the fact to maintain the skin always dried, thereby avoiding the prolonged contact of the same skin with the organic liquid and therefore also the arising of any possible irritation and burning of the same, and the fact that such towels may be so used for longer durations and substituted less frequently, therefore with less interventions of replacement of the devoted personnel, when the patient is in the hospital, and with consequent less purchasing costs of this absorbent material for hygienic-sanitary uses, and elimination of any possible suction machinery foreseen at the present time to perform the same function. Turning back now to the FIG. 1, in the example illustrated therein the container 6 is shaped with a round form adaptable removably into the absorbent towel 7, thereby forming a single body therewith, and remains on this position for prolonged durations, in which the container is filled and emptied many times, until the towel not more hygienically satisfactory becomes useless, and needs to be completely substituted. However, the suction device (container 6 and cock 12) may, probably, be re-used after a cleaning and disinfecting cycle thereof, even with domestic systems, for being used for other cycles. In such manner, the absorbent portion only of the same towel (towel 7 with inserted tampon 9) will be substituted. As alternative thereof, it may be considered the hypothesis to prolong the functional time, by applying containers already “pre-loaded with vacuum condition”, by leaving on the seat the “absorbent portion” of the system, and by reserving the possibility to empty and renew the vacuum condition subsequently.

Or, the suction device foresees an “outer” connection (suction pump for creating the vacuum condition again), by means of devices suitable for this scope.

In the example illustrated by the FIG. 2, the towel 7 and the tampon 9 are still identical to the previous ones, however in this case the container 13 is shaped with a different form and may be applied in addition to the towel 7 and the tampon 9 with the same preceding criteria, also to an absorbent towel 14 shaped with different forms and sizes and performing the same functions. These types of absorbent towels including the present suction device may be advantageously used as towels for more or less severe incontinence forms, or for other hospital or extra-hospital uses, like suction pens applicable in the surgical field or with suction systems of intra and post-operating draining, which aren't connected to complex machineries maintaining the vacuum condition (for example, by means of electric energy), and also used for patients lying into bed and/or “spinalized” patients, for monitoring in a bloodless manner the diuresis (for children and olds), as substitute in many cases of the bladder catheter with prevention of the hospital infections deriving from the bladder catheter, and with various models (anatomic man-woman) with different capacity/form of the container-collector under vacuum condition, which may be included into the same towel, of mono-use or multi-use type (in case of small losses), or as container/collector external to the same towel for patients lying into bed or patients with severe losses. Turning now to the FIGS. 3-8, it is illustrated schematically the present absorbent/suction device, comprising an absorbent towel 7 and an absorbent tampon 9 and a container 6, on different steps of its preparation for being applied to the human body, wherein FIGS. 3 and 4 show the vacuum production steps in the interior of the container 6, FIGS. 5 and 6 show the insertion steps of the container under vacuum condition into the tampon (which is in turn inserted into the towel), and FIGS. 7 and 8 show respectively the device activation steps, by positioning the cock 12 in the suction position.

In particular, in the steps shown in FIGS. 3, 4, 5 and 6, it is noted that the container 6 to be put under vacuum condition is first of all removed (see FIG. 3) from its inserted position into the tampon 9, in which the sleeve 11 of the container 6 is inserted on to a correspondent tampon joining piece 15, which is included in the towel. On the contrary, such container is kept attached to the towel and the tampon, in the case in which such elements are connected permanently to each other. Thereafter, the vacuum condition is created into the container 6 by means of a syringe (with screw joining piece 16). In fact, by connecting the syringe (of 50-100 cc) to the container sleeve/cock 11/12, air is sucked from the container 6, under production of a more or less high vacuum condition depending on the number of suctions of the plunger 16. After the cock 12 has been closed, the vacuum production system, which very simply may be constituted also by a simple syringe of 50-100 cc, may be disconnected and the container 6 is screwed on the appropriate joining piece 15, by inserting the sleeve 11 on the joining piece 15 of the tampon 9, which is inserted into the towel 7, and the cock 12 is displaced on the open position, of adjustable type (see FIG. 6), thereby putting into communication the inner room of the container 6 with the tampon 9, and therefore with the towel 7. In this way, the slow but durable suction of the liquid from the towel 7 to the container 6 starts. It is also to point out that it is possible also to produce the vacuum condition in the interior of the container 6 in manners different than that of the preferable way described above, which ways will be described later on. Subsequently, as visible from FIG. 7, the tampon 9 joined with the container under vacuum condition is re-inserted completely in the towel 7, but different models may provide for also containers external to the towel 7, with or without permanent connection to vacuum production systems. It is confirmed that the suction duration may be changed, by adjusting the passage port of the air volume entering the inner room of the container 6, and therefore also the liquid quantity being sucked by the same container. Such adjustment may be effected by means of the cock 12, which may be displaced into different adjustment positions, in each one of which the valve member controlled by the cock closes from time to time calibrated holes having different diameter, thereby changing such passage port and therefore the suction duration. Advantageously, the actual suction start (also with already open cock) may be controlled not only manually, but may be also timed, depending on the needs, by interposing between the suction device and the towel/tampon suitable substances with plugging function of the passage between these elements.

But, more conveniently, the suction start may occur by means of an external compression on to the connectors by the same substances (for example, salts, sugar, glycerine), by creating effective “salt”, “sugar”, “glycerine” valves, etc. . . . So, by way of example only, with salts, sugars and soaps the suction start occurs when the towel 7 starts to be wet and the liquid (for example urine) dissolves the stopper, or some substances like the glycerine dissolve themselves when the temperature changes, as for example suppository.

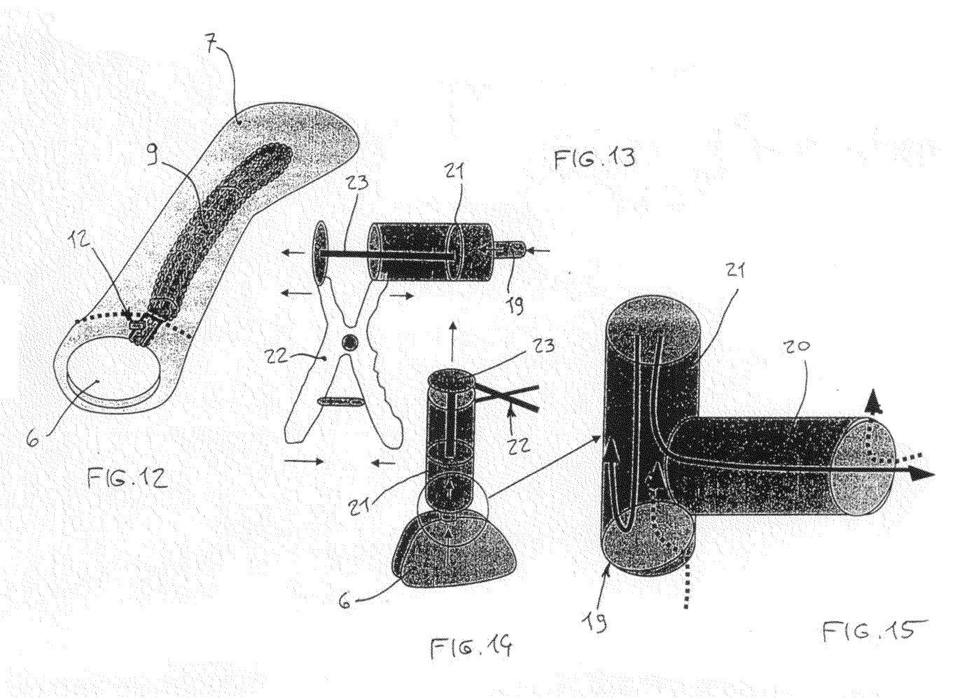

Turning now to the FIGS. 9-11, there are shown different manners to produce the vacuum condition in the interior of the container 6. In particular, from the FIG. 9 it is noted a towel and a tampon like those of the FIG. 1, the container 6 of which is arranged for applying a suction syringe thereto, which syringe has a double opening and closing valve, identified with 18 in the FIGS. 10 and 11, and shaped with a lower restricted portion 19 which may be connected to the sleeve 11 of the container 6 and houses a first movable valve (not indicated), and also shaped with a short sleeve 20 projected laterally from the body of the syringe 21, in which a second movable valve (not indicated too) is housed. Such valves, which constitute a first emptying manner of the container 6, are displaced at the same time the one onto the open position and the other one onto the closed position when the plunger is sliding into the syringe, in a way that when the plunger is extracted the first movable valve opens and the second movable valve closes, and when the plunger is inserted for a subsequent emptying cycle of the container the first movable valve closes and the second movable valve opens, thereby discharging outside the air pumped in the syringe interior. In the second emptying manner of the container 6, illustrated by the FIG. 12, it is noted that a syringe with double opening and closing valve is still utilized, like that illustrated in the FIGS. 9-11, in which as visible from the FIGS. 13, 14 and 15 at least a spring element 22 is used, which is interposed between the plunger 23 and the body of the syringe 21, to facilitate the alternative displacement of the same plunger for emptying the container 6. In the FIGS. 16 and 17 it is now shown another constructive embodiment of the present suction device, which can be used with containers which are laid at the foot of the bed for the patients with catheters after operations. Then, in this case the container 6 is separated from the towel/tampon and laid externally thereto, and is joined to the towel/tampon through a flexible conduit 24, so that the interior of such container under vacuum condition is still put into communication with the towel/tampon by displacing the adjustment cock 12 firstly on the open position, as visible from the FIG. 17, and then on the closed position when the suction is ended. In the FIGS. 18 and 19 there are now shown two further constructive embodiments of the present suction device. In particular, from FIG. 18 it is noted that both the towel/tampon and the container 6, which is applied to the same, are shaped with anatomical forms adaptable onto the part of the human body from which the organic liquid is to be absorbed and sucked, and that the suction force produced by the container 6 may be maintained or increased by means of elastic forces or springs exerting a pull, for example on to the plunger 23 of the syringe 21, or acting in different manners for performing the same function. In the FIG. 19 it is noted that on the same component elements described in the FIG. 18 the suction is produced by means of two small pneumatic cylinders 25 and 26, secured to the container 6 and driven by the compressed air at the same time the one on the suction action and the other one on the compression action, thereby exerting an emptying action of the container with a valve play like that described in the FIGS. 10 and 11. In the FIGS. 20-23 there are now described two possible different manners of applying a suction device according to the invention to the male genital organs, for example in the cases of urinary incontinence. In particular, from the FIGS. 20 and 21 it is noted that the towel/tampon is applied around the penis 27, while the container 6 is fixed onto the towel/tampon upper part. In the FIGS. 22 and 23 it is noted that the towel/tampon is shaped in the form of a cap 28 and applied on to the penis (not shown), and such cap is connected to the container 6 under vacuum condition, in turn connected to the outside through one or more flexible conduits 29 and an outer sucking pump 30.

In the FIG. 24 it is shown an adequately shaped towel/tampon for being adaptable on to the female genital organs, which includes the container 6 under vacuum condition and is also connected to the outside through one or more flexible conduits 31 and an outer sucking pump 32. In the FIGS. 25 and 26 it is shown a suction device according to the invention which is arranged for timing the suction start, and is composed as above described of a substance like for example a salt crystal 33, acting against the flexible connector 34 which connects the container 6 under vacuum condition and the tampon 9, which in this case is bent and housed into a suitable rigid structure 35. Such salt crystal 33 is compressed initially against the connector 34, thereby preventing the liquid to circulate from the tampon 9 to the container 6 under vacuum condition. Then, the liquid leaving the tampon 9 comes into contact with the salt crystal 33, by loosing it progressively, with consequent less squeezing of the connector 34 and progressive opening of the passage port thereof, until such port is completely open when the salt is loosen, and under this condition, in which the cock has been already opened, the suction of the liquid from the tampon 9 toward the container 6 starts. In the FIG. 27 it is shown a suction device according to the invention utilized for a different application thereof, for example to a rag 36 for cleaning and/or remove the stains from floors, surfaces etc., which is connected through the sucking towel/tampon 37 to the container 6 under vacuum condition, see FIG. 27 a, in a manner that the liquid absorbed by the rag 36 is then sucked through the towel/tampon 37. In the FIG. 27 b it is visualized the path of the colour spots (identified as “soil”) which, by virtue of the system absorbent/sucking capacity, from the position of the rag 36 slowly “displace” themselves along the tampon 37, and are collected in the interior of the container 6, this for exemplifying a household/industrial application, for example for cleaning, removing the stains from surfaces, cloths etc.

A further possible application is represented, for example, by a suction device according to the invention (for example, applied to agricultural or not agricultural machines), to collect water of the moisture condensed on to meadows or from wet areas in which water can be collected effectively with difficulty, and only with complex or burdensome systems (on dried areas). Therefore, the invention may be utilized also for the most varied applications, providing suction devices with the above specified characteristics.

In the FIG. 28 it is shown a particular constructive embodiment of the suction device according to the invention, in which it is noted that such suction device 38 is constituted by a set of separated cells 39 under vacuum condition, each one of which may be put under vacuum contemporaneously to the other cells and to this aim it is communicating through a common collector 40 with the above described vacuum production means, in the represented example formed by a syringe system 41, and each cell is also communicating, separately from the other cells, with the tampon 9 housed on a cloth as described above, through respective conduits 42 and suction adjustment means 43, in the example formed by a salt valve or by other substances soluble in presence of the liquid absorbed by the tampon 9 or when the temperature changes, as described previously, in such a manner that the suction start of each cell 39 occurs in presence of the liquid to be sucked contained into the tampon 9 and the suction end occurs when such cell or all the cells are filled with the liquid and the vacuum is exhausted. Besides, this suction device may be further constituted by suitable timer means 44, also communicating with the different cells 39 of the same device, which may be formed by a mechanical timer (example, of the timer-clock type), or an electric timer, a timer supplied by battery, an electronic timer or a timer of other kind, which is adjustable depending on the needs. These timer means, which may be also associated with the above described suction adjustment means 43, put into communication once at a time the different cells 39 with the tampon 9, thereby sucking on different times the liquid from the same tampon and so keeping always dried the cloth onto which such tampon is arranged, and such suction continues until all the cells 39 are filled and the vacuum is exhausted.

Finally, in the FIG. 29 it is shown another possible constructive embodiment of the present suction device, which is substantially constituted like that described in the FIG. 28, and the cells 39 of which are grouped to each other thereby forming a container 44 with multiple cells, even of material of foldable and soft type, having different shapes, which may be applied on different seats, such as for example on a towel 45 put into contact of the skin, together with the absorbent tampon 9. With respect to the embodiment of the FIG. 28, in this case the cells 39 are communicating with the vacuum production means 46 through respective conduits 47 and at least a joining valve 48.

1. Suction device for organic liquids from absorbent towels and tampons and the like for hygienic and sanitary uses and for liquids of other kind for different applications, wherein such towels and tampons are applied on to the parts of the human body emitting such liquids, in order to absorb the same liquids, characterized by container means (6) which can be connected to each absorbent towel/tampon and provided with adjustment means (12), which can be connected to vacuum production means (16, 18, 21, 25, 26) and adapted to be displaced from an open to a closed position thereof, and vice versa, respectively in order to create a high vacuum condition and for maintaining such vacuum condition into said container means (6), said container means (6) being adapted to be put into communication with the environment and the towel/tampon, by displacing said adjustment means (12) in the open position again, thereby producing a suction action of variable duration adapted to suck and convey the liquids, absorbed by said absorbent towel/tampon, into said container means (6), where they are collected and afterwards discharged outside, and with subsequent arrangement of said container means (6) for being put under vacuum condition again, with consequent further suctions and discharges of the liquids with respect to said container means (6).

2. Suction device according to claim 1, characterized in that said container means comprise at least a container (6) with different shapes and sizes, adaptable with respect to said towel/tampon and to the human body parts to which this latter is applied, said container being provided with said adjustment means (12) and shaped for the junction with said towel/tampon.

3. Suction device according to claim 2, characterized in that each towel/tampon (7) is made with different shapes and sizes, depending on the applications to which it is assigned, and the correspondent tampon (9) is inserted therein, which tampon is provided with a joining piece (15) which can be connected removably to said container (6), thereby putting into communication the interior of the same container with the towel/tampon, through said adjustment means (12).

4. Suction device according to claim 2, characterized in that said container (6) is kept attached to the towel/tampon, when these elements are connected to each other in a permanent manner.

5. Suction device according to claim 3, characterized in that said adjustment means comprise at least a cock (12) displaceable from said open position to said closed position, and vice versa, so as to allow or prevent the communication between the interior of said container (6) and the environment and the towel/tampon.

6. Suction device according to claim 5, characterized in that said cock (12) may be displaced on its open position also in different adjustment positions, in each one of which said valve means close from time to time calibrated holes of different diameter, thereby changing the passage port of the air volume entering the interior of said container (6), and consequently also the duration of the suction and the quantity of the liquid sucked by said container (6).

7. Suction device according to claim 6, characterized in that said suction may activated also automatically, by interposing between said container (6) and the towel/tampon suitable substances having a function to plug the port between these elements, which when contacting the liquids loose them gradually (for example salts, sugars, soaps) and in different times, or loose themselves when the temperature changes (ex. glycerine), etc. . . .

8. Suction device according to claim 7, characterized in that said vacuum production means comprise a syringe with screw joining piece (16), the narrower portion (17) of which is connected to the container (6), and the interior of which is put into communication with the interior of said container (6), by displacing said cock (12) in the open position, in a manner that when the plunger of said syringe (16) is extracted, air contained in the interior of said container (6) is sucked, thereby creating the vacuum condition therein, and by maintaining such vacuum condition by displacing such cock (12) in the closed position.

9. Suction device according to claim 7, characterized in that said vacuum production means comprise a sucking syringe with double opening and closing valve (18), shaped with a lower restricted portion (19) which may be connected to said container (6) and houses a first movable valve, and also shaped with a short sleeve (20) projected laterally from the body of the syringe (21), into which a second movable valve is housed, said first and second movable valves being displaceable contemporaneously when the plunger is sliding into the syringe, the one in the open position and the other one in the closed position, and vice versa, in a manner that when the plunger is extracted, on a first emptying cycle of said container (6), said first movable valve opens and said second movable valve closes, and when the plunger is inserted for a subsequent emptying cycle of said container (6), said first movable valve closes and said second movable valve opens, thereby discharging outside air pumped in the syringe interior.

10. Suction device according to claim 9, characterized in that said syringe with double opening and closing valve (18) is provided with resilient means (22) interposed between the plunger (23) and the body of the syringe (21), to facilitate the alternative displacement of the same plunger for emptying said container (6).

11. Suction device according to claim 7, characterized in that said container (6) may be separated from the towel/tampon and laid on externally thereto, and is connected to the towel/tampon through a flexible conduit (24).

12. Suction device according to claim 9, characterized in that the suction force produced by said container (6) may be maintained or increased by elastic means exerting a pull, for example on to the plunger of said syringe (21) during the alternate sliding thereof.

13. Suction device according to claim 9, characterized in that the suction force namely the vacuum condition of said container (6) is produced by means of two small pneumatic cylinders (25, 26) secured to the same container and driven by the compressed air contemporaneously the one in the suction action and the other one in the compression action, with the same alternate displacement of said first and second movable valve.

14. Suction device according to claim 1, characterized in that the towel/tampon may be applied to the male or female genital organs and may be also connected to external pumping means for the production or the renewal of the vacuum condition (30, 32), through respective flexible conduits (29, 31).

15. Suction device according to claim 1, characterized in that it may be utilized also for different applications, for example to one or more rags (36) for cleaning and/or removal of stains from floors, surfaces etc. . . . , which are connected through the towel/tampon to said container (6), in a manner that the liquid absorbed by the rags (36) is then sucked through the towel/tampon.

16. Suction device according to claim 1, characterized in that it may be applied to agricultural machines, to collect water of the moisture condensed on to meadows, or to dried areas in which water can be collected from wet areas only, in absence of effective water sources, the supply of which would require burdensome structures, or it may be applied for the more various applications.

17. Suction device according to claim 1, characterized in that said container means are constituted by a set of separated cells (39) under vacuum condition, each one of which may be put under vacuum contemporaneously to the other cells through a common collector (40), communicating to said vacuum production means (41), and each cell (39) is also communicating, separately from the other cells, to said absorbent towels/tampons (9) through said suction adjustment means (43).

18. Suction device according to claim 17, characterized in that said suction adjustment means (43) may be constituted by at least a salt valve or by other substances soluble in presence of the liquid absorbed by said absorbent towels or tampons (9) or when the temperature changes, in a manner that the suction start by means of each cell (39 occurs in presence of the liquid to be sucked contained into said absorbent towels or tampons (9) and the suction end occurs when such cell or all the cells (39) are filled with the liquid and the vacuum is exhausted.

19. Suction device according to claim 18, characterized in that said suction adjustment means (43) are constituted by timer means (44) of adjustable type according to the needs, also communicating to the different cells (39) and to said absorbent towels or tampons (9) and which in case may be associated also to said salt valve or to other soluble substances, and adapted to put into communication once at a time the different cells (39) with said absorbent towels or tampons (9), thereby absorbing on different times the liquid contained into said absorbent towels or tampons (9), until all the cells (39) are filled and the vacuum is exhausted.

20. Suction device according to claim 19, characterized in that said timer means (44) are formed by a mechanical timer (for example, of the timer-clock type), or an electric timer, a timer supplied by battery, an electronic timer or a timer of other kind.

21. Suction device according to claim 20, characterized in that said cells (39) are grouped to each other, thereby forming a container (44) with multiple cells, even of material of foldable and soft type, having different shapes, which may be applied on different seats, such as for example a towel (45) put into contact of the skin, together with said absorbent towels or tampons (9), said cells (39) of said container (44) being communicating with said vacuum production means (46) through at least a joining valve (48).
